(19) United States
(12) Patent Application Publication
(10) Pub. No.: US 2024/0050079 A1
(43) Pub. Date: Feb. 15, 2024

(54) Delivery System For High Deployment And Recapture Forces

(71) Applicant: BOSTON SCIENTIFIC SCIMED, INC., Maple Grove, MN (US)

(72) Inventors: JOSHUA MARK INOUYE, Brooklyn Park, MN (US); JAMES M. ANDERSON, Corcoran, MN (US)

(73) Assignee: BOSTON SCIENTIFIC SCIMED, INC., Maple Grove, MN (US)

(21) Appl. No.: 18/232,947

(22) Filed: Aug. 11, 2023

Related U.S. Application Data
(60) Provisional application No. 63/397,613, filed on Aug. 12, 2022.

(51) Int. Cl. A61B 17/00; A61B 17/12

(52) U.S. Cl. CPC A61B 17/0057; A61B 17/12122; A61B 2017/00632

(57) ABSTRACT

A delivery system for deploying a medical implant includes a delivery catheter with a lumen, an elongate shaft axially slidably within the lumen, a housing attached to the elongate shaft, and an actuation element disposed within the housing and configured to axially advance and retract the elongate shaft. The actuation element is in direct contact with the elongate shaft and rotation of the actuation element causes axial movement of the elongate shaft.

### CROSS-REFERENCE TO RELATED APPLICATIONS

This application claims the benefit of priority of U.S. Provisional Application No. 63/397,613 filed Aug. 12, 2022, the entire disclosure of which is hereby incorporated by reference.

### TECHNICAL FIELD

The disclosure pertains to medical devices and more particularly to a system and method for delivering an implant into the left atrial appendage of the heart.

### BACKGROUND

A wide variety of medical devices have been developed for medical use including, for example, cardiovascular devices such as implants which are deployed to close off the left atrial appendage. The delivery of some of these medical devices involves high deployment or recapture forces, and conventional axial motion may provide insufficient control over deployment and/or recapture. Of the known medical devices and methods, each has certain advantages and disadvantages. There is an ongoing need to provide alternative medical devices and delivery systems.

### SUMMARY

This disclosure provides design, material, manufacturing method, and use alternatives for medical devices. An example delivery system for deploying a medical implant comprises a delivery catheter having a lumen extending therethrough, an elongate shaft axially slidably within the lumen of the delivery catheter, a housing configured to be removably attached to the elongate shaft, the housing having a first lumen configured to receive the elongate shaft, and an actuation element disposed within the housing and configured to axially advance and retract the elongate shaft.

Alternatively or additionally to the embodiment above, the housing includes a second lumen extending transverse to and intersecting the first lumen, wherein the actuation element includes an actuation shaft extending through the second lumen, the actuation shaft engaging the elongate shaft, wherein rotation of the actuation shaft causes axial movement of the elongate shaft.

Alternatively or additionally to any of the embodiments above, an outer surface of the actuation shaft is textured.

Alternatively or additionally to any of the embodiments above, an outer surface of the actuation shaft is tacky.

Alternatively or additionally to any of the embodiments above, the delivery catheter includes a hub adjacent a proximal end thereof, wherein the housing is configured to be removably attached to proximal portion of the hub.

Alternatively or additionally to any of the embodiments above, the housing includes a first half connected to a second half by a hinge.

Alternatively or additionally to any of the embodiments above, the housing is configured to be attached to the elongate shaft with the actuation shaft already disposed within the second lumen.

Alternatively or additionally to any of the embodiments above, rotation of the actuation shaft causes axial movement of the elongate shaft based only on force of actuation shaft against the elongate shaft.

Alternatively or additionally to any of the embodiments above, the actuation element includes opposing first and second rollers disposed within the housing, the first and second rollers configured to engage opposite sides of the elongate shaft.

Alternatively or additionally to any of the embodiments above, the housing includes first and second recesses configured to retain the first and second rollers, respectively.

Alternatively or additionally to any of the embodiments above, the first and second rollers are cylindrical.

Alternatively or additionally to any of the embodiments above, the first and second rollers each include a concave surface configured to engage the elongate shaft.

Alternatively or additionally to any of the embodiments above, the delivery system further comprises an actuation shaft extending through a second lumen in the housing, the actuation shaft fixed to the first roller such that rotation of the actuation shaft rotates the first roller thereby axially moving the elongate shaft.

Alternatively or additionally to any of the embodiments above, an outer surface of each of the first and second rollers is deformable.

Alternatively or additionally to any of the embodiments above, the housing is rotatable relative to the elongate shaft, wherein the actuation element includes first, second, and third rollers disposed within a recess in the housing, wherein when the housing is disposed on the elongate shaft, rotation of the housing causes the elongate shaft to move axially relative to the housing.

Alternatively or additionally to any of the embodiments above, each of the first, second, and third rollers is mounted to a separate axle.

Alternatively or additionally to any of the embodiments above, each roller disposed at an angle relative to the elongate shaft, wherein first and third rollers are spaced axially apart and are disposed on a first side of the elongate shaft and the second roller is disposed on a second side of the elongate shaft axially between the first and third rollers.

Alternatively or additionally to any of the embodiments above, each of the first, second, and third rollers is tapered.

A further example medical system comprises a left atrial appendage closure device including a self-expandable framework and a proximal hub, a delivery catheter having a lumen extending therethrough sized to receive the left atrial appendage closure device in a collapsed configuration, an elongate shaft axially slidable within the lumen of the delivery catheter, a distal end of the elongate shaft configured to releasably couple with the proximal hub of the left atrial appendage closure device, a housing configured to be removably attached to the elongate shaft adjacent a proximal end of the elongate shaft, the housing having a first lumen configured to receive the elongate shaft, and a second lumen extending transverse to and intersecting the first lumen, and an actuation shaft disposed within the second lumen and in direct contact with the elongate shaft, wherein rotation of the actuation shaft axially advances or retracts the elongate shaft.

Another example delivery system for deploying a medical implant comprises a delivery catheter having a lumen extending therethrough and a hub disposed on a proximal end thereof, an elongate shaft axially slidably within the lumen of the delivery catheter, a distal end of the elongate shaft configured to couple with the medical implant, a housing attached to the hub and rotatable relative to the elongate shaft, the housing having a first lumen configured to receive the elongate shaft, and a plurality of rollers disposed within a recess in the housing and in contact with the elongate shaft, the plurality of rollers configured such that rotation of the housing incrementally axially advances and retracts the elongate shaft.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a graph showing deployment pushing time versus rate/force of deployment;

FIG. 2A is a side cross-sectional view of an example delivery device with a medical implant disposed within a delivery catheter;

FIG. 2B is the delivery device of FIG. 2A with the medical implant expanded distal of the delivery catheter;

FIG. 3 is an end cross-sectional view of the housing shown in FIG. 2A;

FIG. 4 illustrates the housing of FIG. 3 in an open configuration;

FIG. 5 is a side cross-sectional view of another example housing disposed on an elongate shaft;

FIG. 6 is an end cross-sectional view of the housing of FIG. 5 in an open configuration;

FIG. 7 is an end cross-sectional view of the housing of FIG. 5 in a closed configuration;

FIG. 8 is a perspective view of another embodiment of rollers and actuation shaft;

FIG. 9 is a side cross-sectional view of a further example housing disposed on a delivery catheter;

FIG. 10 is a side cross-sectional view of another example housing with another example elongate shaft;

FIG. 11A is a side cross-sectional view of an example gear mechanism for axially moving the drive shaft; and

FIG. 11B is an end view of the gear mechanism of FIG. 10A.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s).

Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Some cardiovascular devices such as left atrial appendage closure devices have designs of the implant or delivery catheter that may require high deployment and/or recapture forces due to downsizing the catheter or adding features that need more space or compression force in order to fit into the delivery catheter. It is desired to minimize the effort needed to deploy the device while retaining the desired device and catheter designs. Furthermore, it is desired that the device may be locked in a certain position to allow steering and manipulation of the device prior to deployment. Mechanisms for increasing mechanical advantage during recapture and deployment may include using rotational rather than axial motion. The use of rotational force may provide an advantage over axial or pushing motion in allowing for smaller, incremental movements.

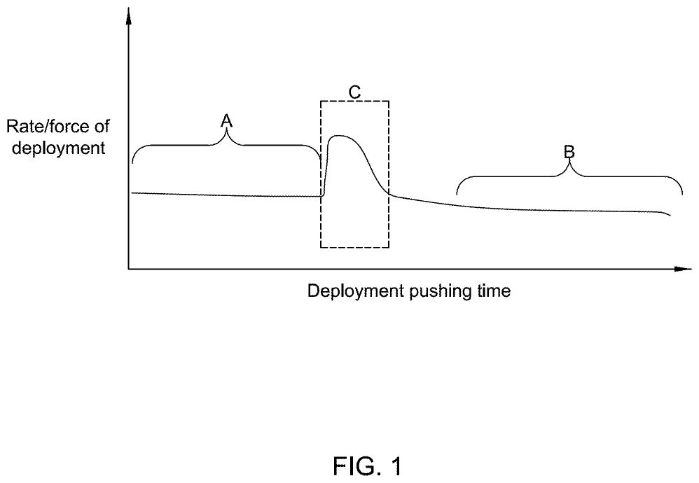

Left atrial appendage closure devices may require high deployment and recapture forces, which may cause a “jump” during deployment, particularly in the few millimeters of axial movement as the device exits the delivery catheter. High recapture and deployment forces on left atrial appendage closure devices may be realized on certain designs as the catheter is downsized for better patient safety or the collapsed device may have larger loaded profile in order to have performance enhancing features. Downsizing the delivery catheter may result in high friction forces during delivery, adding to the jump as the medical device exits the delivery catheter and expands. In some embodiments, a drug coating on all or a part of the medical device may increase the friction between the medical device and inside of the delivery catheter. This increase in force as the medical device exits the delivery catheter may be particularly large for self-expandable devices. In addition, the construction of some left atrial appendage closure devices can cause a rapid and forceful deployment during advancement of the core wire during a few millimeters of deployment, such as when the device pops open. FIG. 1 illustrates a graph of Deployment pushing time on the X axis versus the Rate and/or force of deployment on the Y axis. The time when the device is within the delivery catheter is indicated at A, the time when the device is out of the delivery catheter is indicated at B, and the “jump” or rapid increase in rate and/or force when the device exits the delivery catheter and opens is indicated at C. As can be seen in the graph, while the device remains in the catheter (A), the rate and/or force of deployment remains substantially constant, and similarly when the device is outside the catheter (B), the rate and/or force of deployment remains substantially constant. However, as the device exits the catheter, a sharp increase or “jump” (C) in the rate and/or force of deployment may occur as the implant exits the delivery catheter and expands. This jump in rate and/or force of deployment may damage the delivery device and/or the implant, and may result in an undesirable placement of the implant, requiring repositioning. A solution to this jump in rate and/or force of deployment may be achieved by adding a precision control rotatable actuation element.

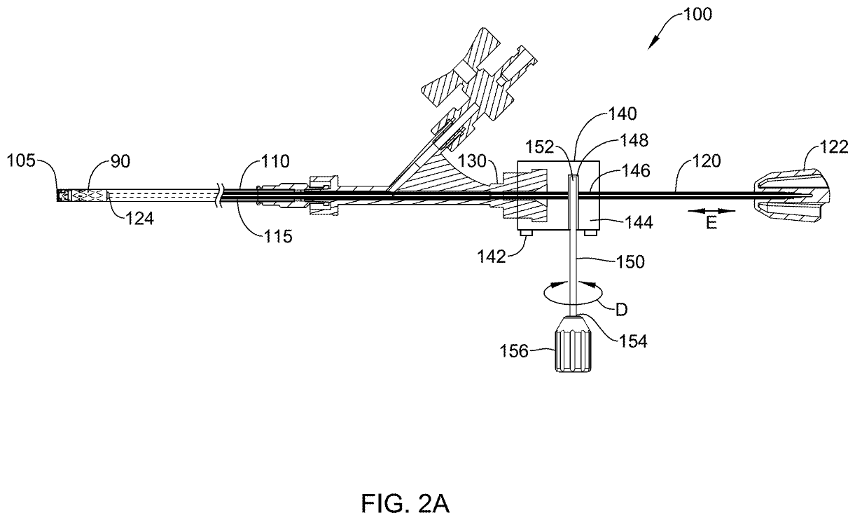

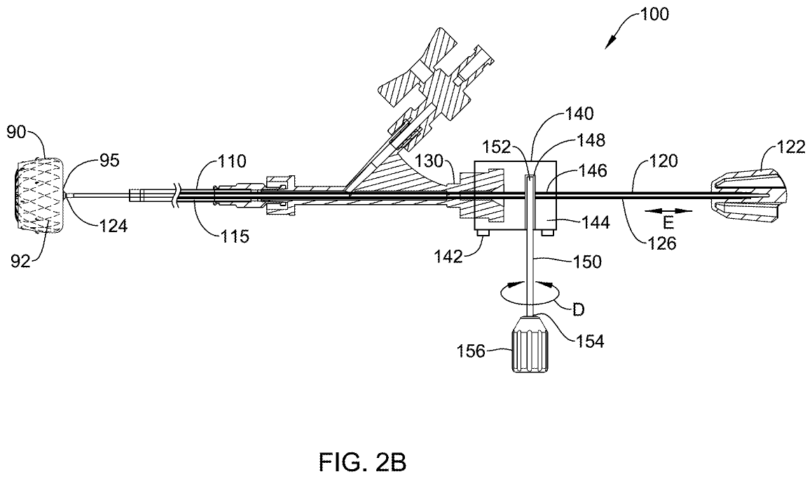

FIG. 2A illustrates a delivery device 100 for deploying a medical implant 90, such as a left atrial appendage closure device. However, it will be understood that the delivery device 100 may be used to delivery any medical device in which incremental axial movement of an elongate shaft is desired at some point during delivery. The delivery device 100 may include a delivery catheter 110 having a lumen 115 extending from a proximal opening to a distal opening 105, an elongate shaft 120 axially slidably within the lumen 115, and a hub 130 disposed on a proximal end of the delivery catheter 110. In use, the medical implant 90 may include a proximal hub 95 configured to be releasably attached to the distal end 124 of the elongate shaft 120. The hub 130 may be part of a luer structure as illustrated, or any other type of hub. The elongate shaft 120 may be longer than the delivery catheter 110 such that proximal region 126 of the elongate shaft 120 extends proximal of the hub 130 when the distal end 124 of the elongate shaft 120 extends distal of the delivery catheter 110 in order to deliver the medical implant 90, as shown in FIG. 2B. The elongate shaft 120 may have a handle 122 disposed on a proximal end thereof, and the distal end 124 may be configured to removably engage the medical implant 90. The handle 122 may be used to push or pull the elongate shaft 120 relative to the delivery catheter 110. In some examples, the elongate shaft 120 may also be rotatable relative to the delivery catheter 110, such as to release the medical implant 90.

In embodiments in which the medical implant is a self-expanding left atrial appendage closure device 90, the left atrial appendage closure device 90 may have an expandable framework 92 configured to shift between a collapsed configuration (e.g., FIG. 2A), wherein the left atrial appendage closure device 90 is disposed within the lumen 115 of the delivery catheter 110 proximate the distal end 124 in the collapsed configuration, and a deployed configuration (e.g., FIG. 2B). The left atrial appendage closure device 90 and/or the expandable framework 92 may be configured to automatically shift between the collapsed configuration and the deployed configuration when the left atrial appendage closure device 90 is disposed distal of the distal end 124 of the delivery catheter 110, and/or when the left atrial appendage closure device 90 is unconstrained by the delivery catheter 110. The left atrial appendage closure device 90 may be disposed at and/or releasably connected to a distal end of the elongate shaft 120.

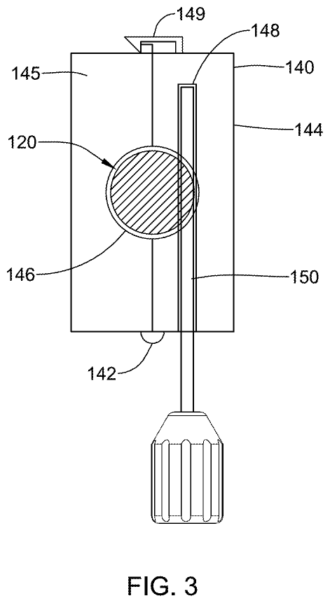

The delivery device 100 may include a housing 140 removably coupled to the elongate shaft 120 adjacent a proximal end of the elongate shaft 120. In some embodiments, the housing 140 may be disposed over at least a proximal portion of the hub 130. The housing 140 may be removably coupled to the hub 130. For example, the housing 140 may have one or more hinges 142 allowing a first portion 144 and a second portion 145 (see FIG. 3) to open and close in a clamshell manner. The housing 140 may have a first lumen 146 extending completely therethrough and configured to receive the elongate shaft 120. The housing 140 may include an actuation element configured to axially advance and retract the elongate shaft relative to the delivery catheter 110. The actuation element may be configured to axially move the elongate shaft incrementally back and forth without any additional elements. In some embodiments, the actuation element may produce fine sub-millimeter axial movement of the elongate shaft 120 while requiring relatively low torque to achieve the axial movement. The actuation element may also be configured to move the elongate shaft completely through the delivery catheter from a first position in which the distal end of the elongate shaft first enters the proximal end of the delivery catheter 110 to a second position in which the handle on the elongate shaft abuts the housing 140.

In the embodiment illustrated in FIGS. 2A and 2B, the first portion 144 of the housing may have a second lumen 148 extending at least partially therethrough and transverse to the first lumen 146 such that the second lumen 148 intersects the first lumen 146. The second lumen 148 is sized and configured to receive an actuation shaft 150 having a first end 152 extending into the second lumen 148, and a second end 154 having a handle 156 or knob. The actuation shaft 150 is configured to engage the elongate shaft 120 with a friction engagement such that rotation of the actuation shaft 150 in either direction, indicated by arrow D, causes axial movement of the elongate shaft 120, indicated by arrow E. The friction engagement allows the rotation of the actuation shaft 150 to cause axial movement of the elongate shaft 120 without any slippage between the actuation shaft 150 and the elongate shaft 120. The degree of rotation of the actuation shaft 150 may be very small, providing the advantage of fine adjustment to the axial movement of the elongate shaft 120. In some embodiments the outer surface of the actuation shaft 150 may be textured. The outer surface of the elongate shaft 120 may also be textured. In one example, at least a portion of the outer surface of the elongate shaft 120 may include gear teeth, and the outer surface of the actuation shaft 150 may include mating gear teeth. In other embodiments, the outer surface of the actuation shaft 150 may be deformable, tacky, sticky, and/or textured. For example, the outer surface of the actuation shaft 150 may be coated with elastomer, silicone, rubber, etc., or may have a raised pattern such as ridges, raised dots, cross-hatching, etc.

FIG. 3 illustrates the housing 140 in side profile attached to the elongate shaft 120, with the details of the hub 130 omitted, showing the first portion 144 and second portion 145 of the housing 140 connected at hinge 142. Opposite the hinge 142 is a closure mechanism 149, such as the illustrated snap fit engagement mechanism. In other embodiments, the closure mechanism 149 may be a friction fit mechanism, snap, clasp, retractable ball lock and release mechanism, etc. In other embodiments, the first portion 144 and the second portion 145 of the housing 140 may be completely separate elements, with closure mechanisms 149 on opposite ends. The housing 140 may be removable from the elongate shaft 120 and hub 130, thus the housing 140 may be used with many existing medical device delivery systems. The housing 140 may be clamped onto the elongate shaft 120 and hub 130 at any point in the delivery procedure, up to just prior to the medical device being moved out of the distal end of the delivery device. As shown, the actuation shaft 150 extends into the first lumen 146 where the actuation shaft 150 engages the elongate shaft 120 within the first lumen 146. The engagement of the actuation shaft 150 with the elongate shaft 120 is seen within the first lumen 146 in FIG. 3. The frictional engagement of the actuation shaft 150 and the elongate shaft 120 allows rotation of the actuation shaft 150 to provide incremental axial movement of the elongate shaft 120, based only on the force of the interaction of the two shafts. This incremental movement of the elongate shaft 120 provides improved control over the axial movement of the elongate shaft 120 and thereby the deployment of the medical device. This incremental movement provides improved control at the point when the medical device exits the delivery catheter and expands, avoiding the “jump” discussed above.

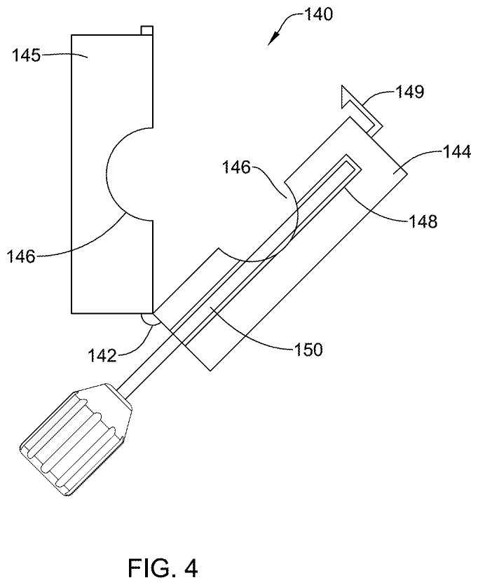

FIG. 4 shows the housing 140 in an open configuration, with the closure mechanism 149 disengaged and the first portion 144 and the second portion 145 separated at their ends opposite the hinge 142. The first lumen 146 that is configured to receive the elongate shaft 120 may be formed partially within the first portion 144 and partially within the second portion 145 of the housing 140. This allows the housing 140 to be clamped onto the hub 130 and the elongate shaft 120 after the elongate shaft 120 is loaded into the delivery catheter 110. The actuation shaft 150 is shown extending within the second lumen 148 and transversely through a region of the first lumen 146. The intersection of the first lumen 146 and the second lumen 148 allows the actuation shaft 150 to directly contact the elongate shaft 120 when the housing 140 is clamped onto the elongate shaft 120. The actuation shaft 150 may be disposed within the second lumen 148 in the housing 140 before attaching the housing 140 to the elongate shaft 120. However, in other embodiments, the actuation shaft 150 may be inserted into the second lumen 148 of the housing 140 after the housing 140 is attached to the elongate shaft 120.

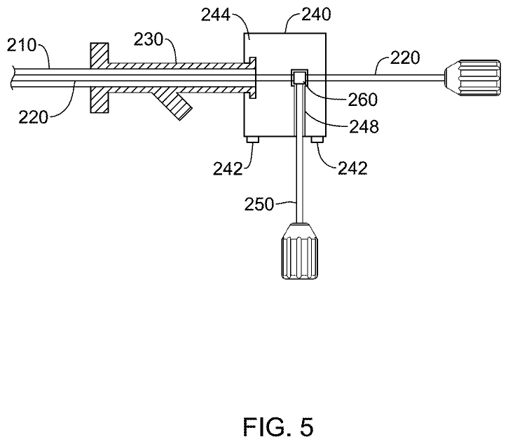

Instead of the actuation shaft 150 directly engaging the elongate shaft 120 illustrated in FIGS. 2A-4, the housing 240 may include an actuation element in the form of opposing first and second rollers 260, 262 configured to engage opposite sides of the elongate shaft 220. See FIGS. 5-7. Similar to the housing 140 illustrated in FIGS. 2A-4, the housing 240 may have first and second portions 244, 245 connected by at least one hinge 242. The first roller 260 may be fixed to an actuation shaft 250 extending through a second lumen 248 in the first portion 244 of the housing. The first roller 260 is controlled by rotating the actuation shaft 250, while the second, opposing roller 262 rotates freely. Each of the first and second roller 260, 262 may reside within a recess 265 in the first and second portion 244, 245 of the housing 240, respectively. The second roller 262 may freely rotate around an axle 261 extending through the recess 265 and attached to the second portion 245 of the housing 240. When the housing 240 is clamped onto the hub 230 and elongate shaft 220, the first and second rollers 260, 262 are pressed into direct contact with the elongate shaft 220, and rotation of the actuation shaft 250 causes the first roller 260 to turn which moves the elongate shaft 220 axially as the second roller 262 rotates freely. As with the elongate shaft 120 described above, the outer surface of the first roller 260 may be textured or may be deformable, tacky, sticky, and/or textured. For example, the outer surface of the first roller 260 may be formed or coated with elastomer, silicone, rubber, etc., or may have a raised pattern such as ridges, raised dots, cross-hatching, etc.

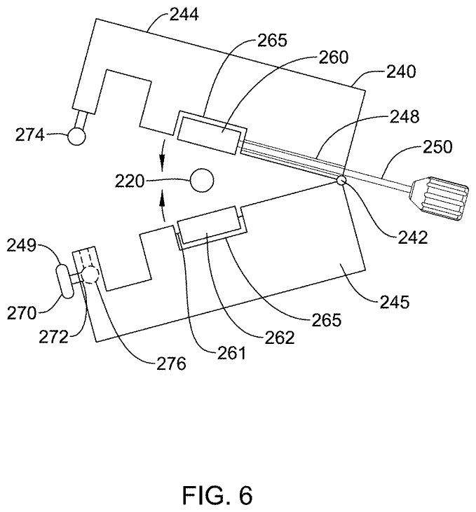

FIG. 6 also illustrates another embodiment of closure mechanism 249 configured to releasably close the first and second portions 244, 245 of the housing 240. In this embodiment, the closure mechanism 249 includes a pin 270 and spring 272 disposed on the second portion 245 of the housing 240, adjacent a recess 276 sized to receive a projection 274 extending from the bottom surface of the first portion 244 of the housing 240. The projection 274 may include an enlarged end that fits into the recess 276 and is secured by the pin 270 when the spring 272 is in a relaxed state. In order to uncouple the first and second portions 244, 245 of the housing 240, the pin 270 is pulled away from the housing 240, compressing the spring 272 and releasing the projection 274. This type of closure mechanism 249 may be included on any of the embodiments of housing described herein, and the closure mechanism 149 discussed above may be provided on housing 240.

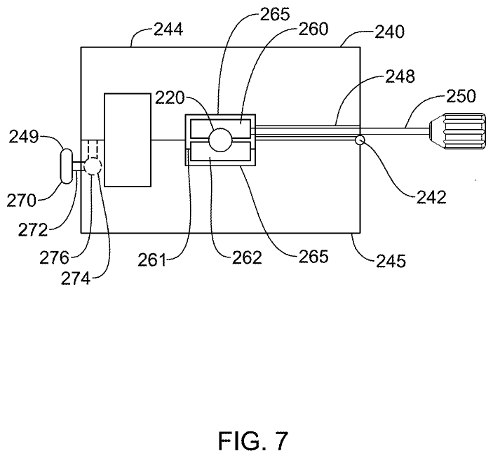

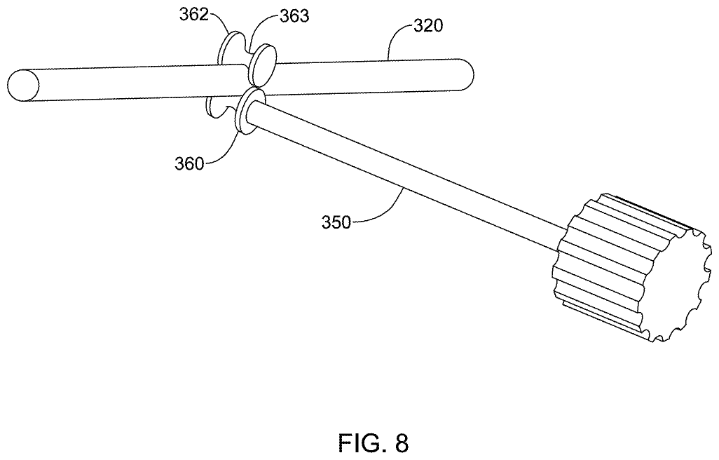

The first and second rollers 260, 262 may be spherical or cylindrical. In some embodiments, the surface of the rollers 260, 262 may be deformable to conform to the surface of the elongate shaft 220 and provide a larger contact area with the elongate shaft 220, as shown in FIG. 7. The surface of each roller 260, 262 that is in contact with the elongate shaft 220 is deformed, providing an increased surface area of contact. In other embodiments, each of the first and second rollers 360, 362 may be spool shaped with a concave surface 363 configured to match the outer surface shape of the elongate shaft 320, as shown in FIG. 8. The actuation shaft 350 may be fixed to the end of the first roller 360 as shown. The concave surface 363 provides a large surface area on each of the rollers 360, 362 in contact with the elongate shaft 320, which may provide precise and incremental axial movement of the elongate shaft 320 by rotating the actuation shaft 350. Additionally, a deformable, sticky/tacky, and/or textured surface on the rollers 360, 362, such as discussed above with regard to the first roller 260, may prevent slipping of the rollers against the elongate shaft 320. The remainder of the housing structure may be as shown in FIGS. 5-7.

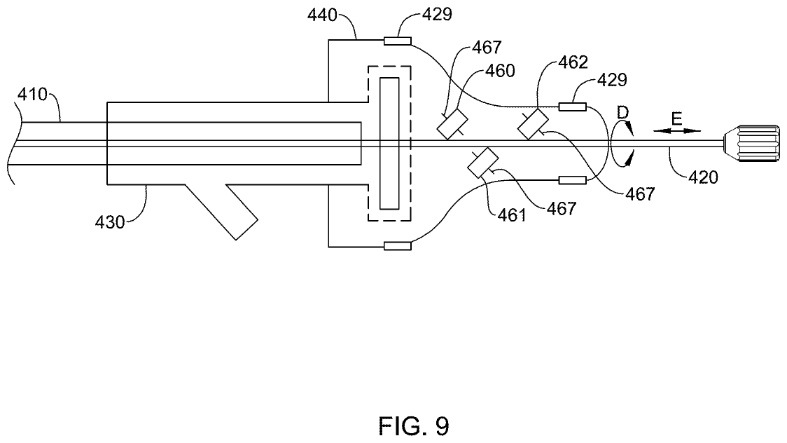

FIG. 9 illustrates another embodiment of housing 440 configured to be rotatably attached to the hub 430 fixed to the delivery catheter 410. The housing 440 may be removably coupled to the proximal end of the hub 430, with the housing 440 having first and second portions and a plurality of closure members 429, as discussed above. The housing 440 may have a first lumen configured to receive the elongate shaft 420. The housing 440 may include an inner recess with three or more rollers 460, 461, 462 each disposed at an angle relative to the elongate shaft 420 and in contact with the elongate shaft 420. The housing 440 is configured to rotate in either direction relative to the elongate shaft 420, as indicated by arrow D. The rollers 460, 461, 462 may each disposed on a separate axle 467 fixed to the housing 440. The rollers 460, 461, 462 each frictionally engage the elongate shaft 420 and axially move the elongate shaft 420, as indicated by arrow E, as the housing 440 is rotated. The first roller 460 and third roller 462 may be spaced axially apart and disposed on a first side of the elongate shaft 420 and the second roller 461 may be disposed on a second side of the elongate shaft 420 axially between the first and third rollers 460, 462. In some embodiments, the first, second, and third rollers 460, 461, 462 may have a tapered shape, such as frustoconical. This may provide a larger contact surface with the elongate shaft 420.

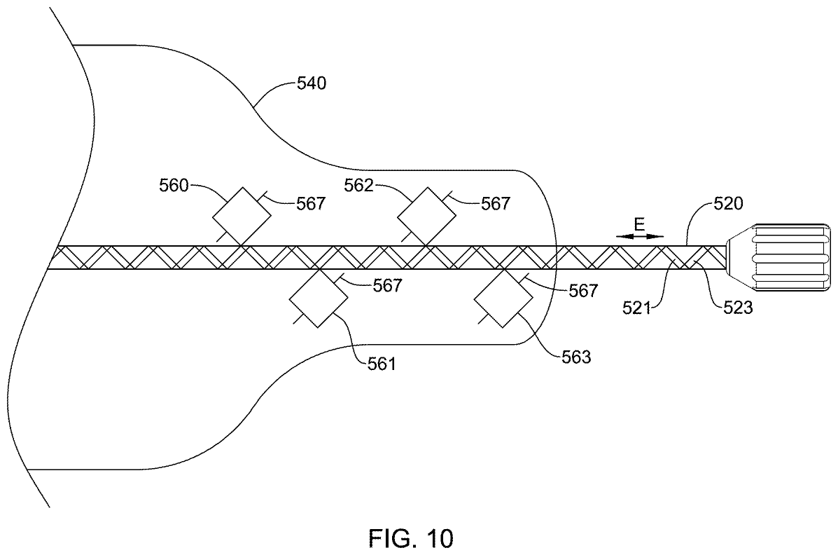

FIG. 10 illustrates another example housing 540 configured to be rotatably attached to the hub with a modified elongate shaft 520. Housing 540 is illustrated with four rollers 560, 561, 562, 563 disposed on axles 567 fixed to the housing 540. In this embodiment, the elongate shaft 520 has a double opposing helical groove structure including a first helical groove 521 extending in a first direction, and a second helical groove 523 extending in a second direction opposite the first direction. The rollers 560, 561, 562, 563 may be angled such that an edge of each roller engages one of the first and second helical grooves 521, 523. The engagement of the rollers 560, 561, 562, 563 and first and second helical grooves 521, 523 may increase the friction force driving the axial movement, indicted by arrow E, of the elongate shaft 520 as the housing 540 is rotated around the elongate shaft 520. Similar to the housing 440, the housing 540 may include closure members and a recess for receiving the proximal end of the hub (not shown).

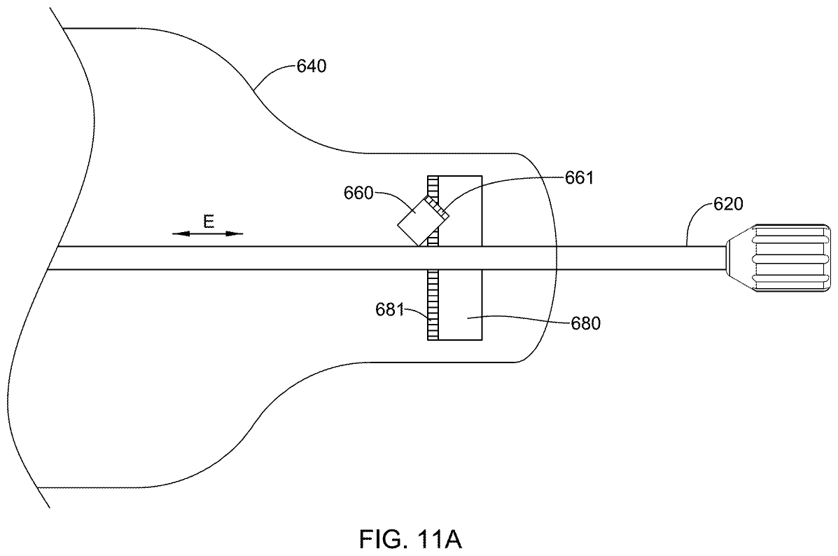

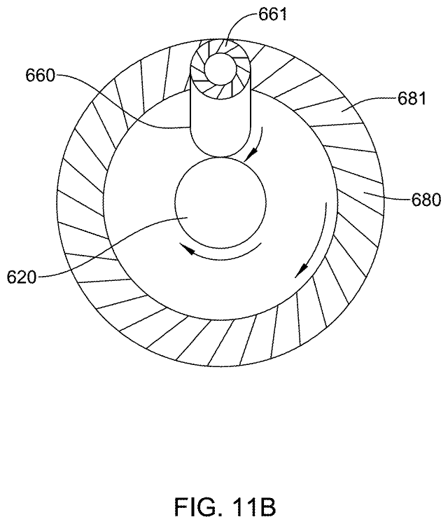

In a further embodiment, an auxiliary gear, such as a planetary gear concentric with the elongate shaft, may drive the rollers, as shown in FIGS. 11A and 11B. FIG. 11A is a side cross-sectional view of an elongate shaft 620 extending through a housing 640. A single roller 660 is shown engaging the elongate shaft 620. It will be understood that additional rollers may be provided. A planetary gear 680 concentric with the elongate shaft 620 may be coupled to the housing 640, such that rotation of the housing 640 rotates the planetary gear 680. See FIG. 11B. The planetary gear may have beveled teeth 681 that engage beveled teeth 661 on a roller 660 that engages both the planetary gear 680 and the elongate shaft 620. Rotation of the housing 640 may cause rotation of the planetary gear 680, which rotates the roller 660 which moves the elongate shaft 620 rotationally and axially, as indicated by arrow E. As shown by the arrows in FIG. 11B, the planetary gear 680, roller 660, and elongate shaft 620 all rotate in the same direction. The elongate shaft 620 rotates as it moves axially.

It will be understood that materials that can be used for the various components of the housing 140, 240, 340, 440, 540, 640 and actuation element (and/or other systems or components disclosed herein) and the various elements thereof disclosed herein may include those commonly associated with medical devices. For simplicity purposes, the following discussion makes reference to the housing 140 (and variations, systems or components disclosed herein). However, this is not intended to limit the devices and methods described herein, as the discussion may be applied to other elements, members, components, or devices disclosed herein.

In some embodiments, the housing 140 (and variations, systems or components thereof disclosed herein) may be made from a metal, metal alloy, ceramics, zirconia, polymer (some examples of which are disclosed below), a metal-polymer composite, combinations thereof, and the like, or other suitable material.

In some embodiments, the housing 140 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A delivery system for deploying a medical implant, comprising:
a delivery catheter having a lumen extending therethrough;
an elongate shaft axially slidably within the lumen of the delivery catheter;
a housing configured to be removably attached to the elongate shaft, the housing having a first lumen configured to receive the elongate shaft; and
an actuation element disposed within the housing and configured to axially advance and retract the elongate shaft.

2. The delivery system of claim 1, wherein the housing includes a second lumen extending transverse to and intersecting the first lumen, wherein the actuation element includes an actuation shaft extending through the second lumen, the actuation shaft engaging the elongate shaft, wherein rotation of the actuation shaft causes axial movement of the elongate shaft.

3. The delivery system of claim 2, wherein an outer surface of the actuation shaft is textured.

4. The delivery system of claim 2, wherein an outer surface of the actuation shaft is tacky.

5. The delivery system of claim 1, wherein the delivery catheter includes a hub adjacent a proximal end thereof, wherein the housing is configured to be removably attached to proximal portion of the hub.

6. The delivery system of claim 1, wherein the housing includes a first half connected to a second half by a hinge.

7. The delivery system of claim 2, wherein the housing is configured to be attached to the elongate shaft with the actuation shaft already disposed within the second lumen.

8. The delivery system of claim 2, wherein rotation of the actuation shaft causes axial movement of the elongate shaft based only on force of the actuation shaft against the elongate shaft.

9. The delivery system of claim 1, wherein the actuation element includes opposing first and second rollers disposed within the housing, the first and second rollers configured to engage opposite sides of the elongate shaft.

10. The delivery system of claim 9, wherein the housing includes first and second recesses configured to retain the first and second rollers, respectively.

11. The delivery system of claim 9, wherein the first and second rollers are cylindrical.

12. The delivery system of claim 9, wherein the first and second rollers each include a concave surface configured to engage the elongate shaft.

13. The delivery system of claim 9, further comprising an actuation shaft extending through a second lumen in the housing, the actuation shaft fixed to the first roller such that rotation of the actuation shaft rotates the first roller thereby axially moving the elongate shaft.

14. The delivery system of claim 9, wherein an outer surface of each of the first and second rollers is deformable.

15. The delivery system of claim 1, wherein the housing is rotatable relative to the elongate shaft, wherein the actuation element includes first, second, and third rollers disposed within a recess in the housing, wherein when the housing is disposed on the elongate shaft, rotation of the housing causes the elongate shaft to move axially relative to the housing.

16. The delivery system of claim 15, wherein each of the first, second, and third rollers is mounted to a separate axle.

17. The delivery system of claim 15, wherein each roller is disposed at an angle relative to the elongate shaft, wherein the first and third rollers are spaced axially apart and are disposed on a first side of the elongate shaft and the second roller is disposed on a second side of the elongate shaft axially between the first and third rollers.

18. The delivery system of claim 17, wherein each of the first, second, and third rollers is tapered.

19. A medical system comprising:
a left atrial appendage closure device including a self-expandable framework and a proximal hub;
a delivery catheter having a lumen extending therethrough sized to receive the left atrial appendage closure device in a collapsed configuration;
an elongate shaft axially slidable within the lumen of the delivery catheter, a distal end of the elongate shaft configured to releasably couple with the proximal hub of the left atrial appendage closure device;
a housing configured to be removably attached to the elongate shaft adjacent a proximal end of the elongate shaft, the housing having a first lumen configured to receive the elongate shaft, and a second lumen extending transverse to and intersecting the first lumen; and
an actuation shaft disposed within the second lumen and in direct contact with the elongate shaft, wherein rotation of the actuation shaft axially advances or retracts the elongate shaft.

20. A delivery system for deploying a medical implant, comprising:
a delivery catheter having a lumen extending therethrough and a hub disposed on a proximal end thereof;
an elongate shaft axially slidably within the lumen of the delivery catheter, a distal end of the elongate shaft configured to couple with the medical implant;
a housing attached to the hub and rotatable relative to the elongate shaft, the housing having a first lumen configured to receive the elongate shaft; and
a plurality of rollers disposed within a recess in the housing and in contact with the elongate shaft, the plurality of rollers configured such that rotation of the housing incrementally axially advances and retracts the elongate shaft.
